(19) United States
(12) Patent Application Publication
(10) Pub. No.: US 2005/0249720 A1
(43) Pub. Date: Nov. 10, 2005

(54) Cosmetic skin formulation

(76) Inventor: Mary Rose Perez, Austin, TX (US)

Correspondence Address: Charles D. Gunter, Jr.;Whitaker, Chalk, Swindle & Sawyer, LLP, Suite 3500, 301 Commerce Street, Fort Worth, TX, 76102-4186, US

(21) Appl. No.: 11/121,449

(22) Filed: May 4, 2005

Related U.S. Application Data
(60) Provisional application No. 60/568,430, filed on May 5, 2004.

(51) Int. Cl. A61K 38/46; A61K 35/64; A61K 35/78

(52) U.S. Cl. 424/94.65; 424/758; 424/777; 424/771; 424/539

(57) ABSTRACT

A cosmetic formulation is shown which includes as a blend of ingredients natural honey as an antiseptic and a blend of natural fruits including pineapple, mango and papaya as active dermal exfoliating agents. The blend also includes at least two melons as buffering agents for the active exfoliating agents.

### CROSS REFERENCE TO RELATED APPLICATIONS

The application claims priority from provisional application Ser. No. 60/568,430, filed May 5, 2004, entitled “Cosmetic Formulation.”

### BACKGROUND OF THE INVENTION

1. Field of the Invention

The present invention relates generally to formulations for treating the human dermis and, more specifically, to a cosmetic formulation made from a blend of honey, natural fruits and natural fruit enzymes and to a method of using the same to exfoliate the skin, to help maintain the youthfulness of the skin and to counter the deleterious effects of free radical deterioration of the skin.

3. Description of the Prior Art

The deleterious effects of free radicals on the human dermis have been documented to some extent, but are also continuing to be investigated. It is suspected that one of the causes of the deterioration of skin over time is the formation of free radicals within the elements forming the skin, such as the various fibers and cells. The formation of such free radicals may be caused, at least in part, by the action of external radiation of sunlight. The free radicals thus formed may attack the skin elements through a chain reaction that continues as long as the free radical exists. This action can continue until, e.g., the free radical combines with a so-called scavenger or inhibitor that acts to produce a stable molecule. The adverse action of free radicals at the molecular level of the skin may cause destruction of cell DNA and an erosion of the connective tissues themselves (collagen and elastin). The result is an aging appearance in the skin.

Various types of cosmetic and dermatological treatments are available to rejuvenate aging or damaged skin. In many cases, an exfoliator is used to remove dead skin cells, thereby exposing the living layers. These treatments provide a smoother and softer, more youthful appearance. Exfoliating treatments also allow the living layers to grow and stay healthy. However, at the present time, many exfoliating compositions use abrasive elements or use harsh burning or acidic compounds to remove dead skin cells from the face. The burning or overly acidic components of certain of the existing exfoliants can break small capillaries in the skin or otherwise cause irritation in the dermis. Thus, while the overall beneficial effects of exfoliation treatments are understood, many of the presently existing cosmetic compositions which provide an exfoliating effect are too harsh, or require an abrasive component which could further irritate the skin.

Since the general theory of skin damage due to environmental factors is understood at least in part, substances that interrupt or inhibit the formation of free radicals during the free radical chain reaction are of great interest in the preparation of cosmetic and cosmetic/dermatological compositions, including exfoliating compositions. One area of interest at the present time is in the area of natural and artificially produced enzymes. The human dermis is a rich reservoir of enzyme activity. There are skin care formulations which aim to enhance or inhibit the activity of natural enzymes in the skin There are also skin care products which contain enzymes which purportedly work with the natural enzymes in the skin to help maintain the youthfulness of the skin. Today, there is increasing interest in fruit enzymes as a source of skin care agents which provide restorative dermatological effects.

At a recent meeting of the American Association For Cancer Research (Second Annual International Conference On Frontiers In Cancer Prevention Research, Phoenix, Ariz., 2003), studies were reported in which the effects of three different types of fruit extracts were examined in relation to preventing sun-related skin damage in animal and laboratory testing. Specifically, the researchers were looking at the effect of using extracts of pomegranate, grape and citrus fruits in offering some form of sun damage protection. While the proactive potential of such extracts appears evident, the studies were preliminary and an attempt “to evaluate the mechanistic effects of these food ingredients.”

In this regard, U.S. Pat. No. 5,916,576, issued Jun. 29, 1999, describes a free radical scavenger in the form of orange extract which is substantially free of vitamin C. That same reference discusses a number of prior art publications including French Demande 2, 597, 337 A2 which describes an anti-aging cosmetic which includes as a source of vitamin C acerola cherry extract and a second fat soluble ingredient which includes such scavengers as vitamin E. Also, Japanese laid open applications 2-200610 and 70-61915 describe an extract obtained from fruits of acerola may be incorporated into cosmetics. The applications describe the presence of L-ascorbic acid which produces a whitening effect in the skin.

U.S. Pat. No. 5,705,166, issued Jan. 6, 1998, describes an exfoliating cream which includes unripened papaya juice and honey. The pH value of the formulation is altered, as by adding baking soda, to “activate” the composition.

U.S. Pat. No. 6,630,163, issued Oct. 7, 2003, describes a method of treating dermatological disorders with fruit extracts, and particularly with extract of pomegranate. The extracts are provided in a pharmaceutically acceptable carrier. The compositions may also contain a transition metal component such as zinc.

The above excerpts from the literature reveal a need for a cosmetic formulation and for a system of skin care which can be applied to the human epidermis to renew aging skin, reduce pores and improve the overall appearance of the outer skin layers while creating a smoother texture.

A need also exists for such a formulation which will counteract sun damage to the skin, returning the skin to a normal, healthy level.

A need also exists for such a formulation which will exfoliate the skin without unduly irritating the dermis, thereby providing a restorative dermatological effect.

### SUMMARY OF THE INVENTION

The cosmetic formulations of the invention are based upon a blend of ingredients which include at least natural honey for its soothing and antiseptic qualities; a blend of natural fruits including pineapple, mango and papaya as active dermal exfoliating agents; and a blend of at least two melons as buffering agents for the active dermal exfoliating agents. Preferably, the blend of at least two melons includes a red fruit melon and a pink/orange fruit melon such as water melon and cantaloupe melon. The preferred formulation is a blend of extracts of the ingredients in the formulation. The formulation can be provided as a powdered blend which includes extracted enzymes of at least selected ones of the ingredients of the formulation. In one preferred form, the formulation is a powdered blend of powdered honey, powdered fruit enzymes and powdered fruits. The fruit enzymes can include papain enzyme, bromelain enzyme and catalase enzyme.

A method of treating the human dermis is also disclosed. A cosmetic formulation is first prepared of natural powdered honey and powdered fruit extracts, the cosmetic formulation comprising:
natural honey as an antiseptic and soothing agent;
a blend of natural fruits including pineapple, mango and papaya as active dermal exfoliating agents;
a blend of at least two melons as buffering agents for the active exfoliating agents;
wherein the formulation is a powdered blend which includes extracted enzymes of at least selected ones of the ingredients of the formulation.

Preferably, the formulation is furnished in the form of a dry powdered cosmetic formulation containing the raw enzyme and fruit extracts. The formulation is prepared for use by mixing with water to form a paste which is then applied to the human dermis.

Additional objects, features and advantages will be apparent in the written description which follows.

### DETAILED DESCRIPTION OF THE INVENTION

The cosmetic formulations of the invention include natural honey for its soothing and antiseptic qualities. Raw honey is a natural moisturizer for the skin and soothes the effect of the other enzyme constituents of the present cosmetic formulations on the skin. The natural honey is present in the cosmetic formulations of the invention in the range from about 5 to 15% by weight, based upon the total weight of the cosmetic formulation.

The remaining 85 to 95% by weight of the formulations of the invention is comprised of a blend of natural fruits including pineapple, mango and papaya as active dermal exfoliating agents; and a blend of at least two melons as buffering agents for the active dermal exfoliating agents.

A particularly preferred formulation of the invention comprises:
from about 5 to 15% by weight, based upon the total weight of the formulation of natural honey as an antiseptic and soothing agent;
from about 20 to 30% by weight pineapple, from about 5 to 15% by weight mango and from about 15 to 25% by weight papaya as active dermal exfoliating agents;
from about 25 to 35% by weight water melon and from about 5 to 15% by weight cantaloupe melon as buffering agents for the active dermal exfoliating agents.

While the formulations can be prepared as a simple fruit/honey blend and applied to the skin, the preferred method of preparation of the formulations of the invention is to provide the formulation in the form of a dry powder containing powdered honey, powdered fruit and powdered fruit enzymes. The particular fruit enzymes which are extracted are generally known in the relevant arts. However, the particular combination of ingredients in Applicant's above described formulation has a cooperative and synergistic effect which achieves the objects of the present invention.

One of the primary exfoliating agents present in Applicant's formulation is papain. Papain is a protein-digesting enzyme which makes an excellent exfoliant and is found in the fruit and leaves of papaya. Papain can be obtained from dried papaya latex. However, papain can also be obtained from fresh papaya fruit in high concentrations. For purposes of the present invention, the papain is preferably obtained from fresh papaya fruit which is fully ripened.

Papain becomes active at a relatively neutral pH, and is increasingly inactive as the pH is raised or lowered. When the enzyme is active, it is effective in selectively dissolving dead skin cells. However, when active, the enzyme will also very quickly degrade and become ineffective. It is for this reason, among others, that the enzymes used in the formulations of the invention are provided as dried extracts. The problem of degradation upon activation, as well as providing a prolonged shelf life, are eliminated since the compositions of the invention are dry powders until mixed with water to form a “paste” which is immediately applied to the face or other affected area.

In order to prepare the dry extract, fully ripened papaya is used, because it contains a high concentration of papain and helps preserve the enzyme by keeping it inactive. The extract initially has an acidic pH with a value on the order of 4.0. Since the extract is so acidic, it could cause skin burns or irritation in the raw state. It is the combination of fruit enzymes with the particular fruit extracts described above which prevents burning or skin irritation in Applicant's formulations. The pH range of the ultimate product is preferably in the range from about 5.0 to 8.0. Whereas other known enzyme systems can, in some circumstances actually be harmful and exfoliate out of control, Applicant's natural cosmetic formulations offer a unique approach in exfoliating under controlled conditions. Additionally, the raw honey present in Applicant's formulations acts as a natural moisturizer for skin and also soothes the irritating effect of the enzyme on skin.

The following description of the method of producing the dry, powder extract of papain can also be utilized generally in extracting the other fruit enzymes which are utilized in the formulations of the invention.

A quantity of fully ripened papayas are used. The fully ripened papayas are cleaned and deseeded before being pressed in a pressing device which extracts the juice, leaving behind a fine pulp residue. The extract or papaya juice, is then placed in an evaporating chamber or oven. Heating at a preselected temperature for a preselected time slowly evaporates the water content of the pulp residue and produces a concentrated material which can be dried into a powder. It is important that the temperature in the evaporating chamber not exceed about 120 degrees F. (49 degrees C.) in order to not degrade the enzyme.

While the above technique has been used successfully in producing a dry, powdered extract, it will be appreciated by those skilled in the art that other techniques may be utilized as well including freeze drying, vacuum drying and other methods familiar to those experienced in the art of drying fruits and vegetables.

The extraction of the other enzymes is preferably accomplished using the same general techniques. For example, bromelain is a protein-digesting enzyme found in pineapple and also acts as an active exfoliating agent in the formulations of the invention. It can be obtained from pineapple juice by precipitation with acetone and also with ammonium sulfide commercially. Preferably, it is extracted from the fresh fruit in the same general manner as papain described above for the purposes of the present invention.

The final principle enzyme constituent used in the formulations of the invention is catalase. The catalase enzyme is a well known plant enzyme which converts hydrogen peroxide into oxygen and water. It is found in plant material such as the fruits described herein and also in such vegetables as potatoes. The catalase constituent of the present formulations does not come from any one particular contributor, but comes from the combination of specified natural plant ingredients.

The formulations of the invention also preferably include the extract of at least two melons which provide a buffering action. Preferably, the blend of at least two melons includes a red fruit melon and a pink/orange fruit melon. The preferred fruit melons are water melon as the red melon and cantaloupe as the pink/orange melon. The extract of the melons is thought to provide a buffering action which, together with the other ingredients, provides a mild pH of about 7.0 or above for the overall formulation.

The formulations of the invention can contain, as an optional ingredient, a small quantity of willow bark for its salicylate content in order to provide an aspirin-like effect. The willow bark would be present in the range from about 0.5-5.0% by weight of the cosmetic formulation.

As explained above, the preferred formulation is a blend of extracts of the ingredients in the formulation with the most preferred formulation being a powdered blend which includes extracted enzymes of at least selected ones of the ingredients of the formulation. In the most preferred form, the formulation is a powdered blend of powdered honey, powdered fruit enzymes and powdered fruits. The fruit enzymes include the papain enzyme, bromelain enzyme and catalase enzyme, as described above.

On example formulation is prepared by combining the following:

| | |
|---|---|
| red (water) melon | 45-60 oz. |
| papaya | 35-45 oz. |
| mango | 10-20 oz. |
| honey | 10-20 oz. |
| pineapple | 30-50 oz. |
| pink/orange melon | 10-20 oz. |

The above formulation of raw fruits and honey was extracted as discussed above to produce a dry powdered extract which was then packaged in 20 gram packets of dry powder. Alternatively, the dry powdered extract can be divided and offered in approximate 5 gram “per treatment” packages.

The compositions of the invention are extracted from the raw fruit but are then “inactivated” in that they are reduced to the dry, powdered form. The compositions of the invention are “activated” by mixing with water at the time of use and by then applying the resulting paste to the human skin, such as the face region. There is no additional or extra chemical “activator” utilized with the compositions of the invention.

If desired, the dry powdered extract can also contain a further “inactivator” in the form of about 1.0-3.0% by weight of extract of boric acid. The boric acid would insure that no moisture, for example present from the packaging step, was present to prematurely activate the enzyme constituents.

The action by which exfoliation occurs is a known phenomenon discussed in the medical and dermatological texts. Briefly stated, the enzymes are immediately presented to the cornified epithelial layer of the skin and initiate exfoliation. This process results in removal of the dead cells of the cornified epithelium as well as cell remnants, debris and components of the extracellular matrix of the surface layers. This material, which is removed, contains oxidized collagen, and other extracellular matrix (ECM) materials such as hyaluronate derivatives. It is this general action which leaves the skin new looking, smoother and restored in health.

Exfoliation by the use of the fruit enzymes of the invention is effective in cleansing of the skin and in the removal of dead cells of the cornified epithelium. In addition to removing the dead cells from normal skin sloughing, this process removes oxidatively damaged proteins, glycosaminoglycans and other macromolecules of the ECM. This is particularly important in case of damaged skin such as where the skin is damaged by exposure to the sun.

A potential disadvantage of traditional exfoliation processes is that the skin is left with the upper layers of cells exposed, thinned and subject to damage from the environment such as increased sun sensitivity. However, in the case of the cosmetic formulations of the invention, this does not occur. This is due to several different factors. First, the exfoliation takes place at a mild pH which generally exceeds 7.0, thereby minimizing trauma to the skin. Second, the buffering components in the fruit extracts such as the melon extracts buffer the otherwise harsh effects of the active exfoliating agents and thereby promote healing to the tissue and also minimize inflammation of the exfoliated skin. The honey present in the formulation also acts to soothe the affected areas.

An invention has been provided with several advantages. The formulations of the invention contain natural ingredients yet exfoliate the epidermis to remove dead tissue, to reduce pores, renew the skin and create a smoother textured skin. The formulations provide improved properties and buffer the effect of the otherwise harsh active exfoliating agents present in the fruit mixtures. The combination of natural honey with the particular fruit extracts described herein produces a synergistic effect in achieving the desired results mentioned above. The products achieve exfoliation accompanied by soothing and hydration of the skin.

While the invention has been shown in only one of its forms, it is not thus limited but is susceptible to various changes and modifications without departing from the spirit thereof.

1. A cosmetic formulation, comprising as a blend of ingredients:
natural honey as an antiseptic;
a blend of natural fruits including pineapple, mango and papaya as active dermal exfoliating agents;
a blend of at least two melons as buffering agents for the active exfoliating agents.

2. The cosmetic formulation of claim 1, wherein the blend of at least two melons includes a red fruit melon and a pink/orange fruit melon.

3. The cosmetic formulation of claim 2, wherein the blend of the at least two melons includes water melon and cantaloupe melon.

4. The cosmetic formulation of claim 3, wherein the formulation is a blend of extracts of the ingredients in the formulation.

5. The cosmetic formulation of claim 4, wherein the formulation is a powdered blend which includes extracted enzymes of at least selected ones of the ingredients of the formulation.

6. The cosmetic formulation of claim 5, wherein the formulation is a powdered blend of powdered honey, powdered fruit enzymes and powdered fruits.

7. The cosmetic formulation of claim 6, wherein the fruit enzymes include papain enzyme, bromelain enzyme and catalase enzyme.

8. The cosmetic formulation of claim 1, further comprising willow bark as an additional ingredient.

9. A cosmetic formulation, comprising dry powdered extracts of the following ingredients:
natural honey as an antiseptic;
pineapple, mango and papaya as active dermal exfoliating agents;
water melon and cantaloupe melon as buffering agents for the active dermal exfoliating agents.

10. The cosmetic formulation of claim 9, wherein the formulation is a powdered blend which includes extracted enzymes of at least selected ones of the ingredients of the formulation.

11. The cosmetic formulation of claim 10, wherein the formulation is a powdered blend of powdered honey, powdered fruit enzymes and powdered fruits.

12. The cosmetic formulation of claim 11, wherein the fruit enzymes include papain enzyme, bromelain enzyme and catalase enzyme.

13. A cosmetic formulation, comprising dry powdered extracts of the following ingredients:
from about 5 to 15% by weight, based upon the total weight of the formulation of natural honey as an antiseptic;
from about 20 to 30% by weight pineapple, from about 5 to 15% by weight mango and from about 15 to 25% by weight papaya as active dermal exfoliating agents;
from about 25 to 35% by weight water melon and from about 5 to 15% by weight cantaloupe melon as buffering agents for the active dermal exfoliating agents.

14. A method of treating the human dermis, comprising the steps of:
preparing a cosmetic formulation of natural powdered honey and powdered fruit extracts, the cosmetic formulation comprising:
natural honey as an antiseptic;
a blend of natural fruits including pineapple, mango and papaya as active dermal exfoliating agents;
a blend of at least two melons as buffering agents for the active exfoliating agents;
wherein the formulation is a powdered blend which includes extracted enzymes of at least selected ones of the ingredients of the formulation;

15. The method of claim 14, wherein the blend of at least two melons includes a red fruit melon and a pink/orange fruit melon.

16. The method of claim 15, wherein the blend of the at least two melons includes water melon and cantaloupe melon.

17. The method of claim 16, wherein the formulation includes powdered honey, powdered fruit enzymes and powdered fruits.

18. The method of claim 17, wherein the fruit enzymes include papain enzyme, bromelain enzyme and catalase enzyme.

19. The method of claim 18, wherein the dry powdered cosmetic formulation is prepared for use by mixing with water to form a paste which is then applied to the human dermis.
